(19) United States
(12) Patent Application Publication
(10) Pub. No.: US 2018/0044347 A1
(43) Pub. Date: Feb. 15, 2018

(54) CRYSTALLINE SALTS OF HYDROCODONE BITARTRATE

(71) Applicant: GRÜNENTHAL GMBH, Aachen (DE)

(72) Inventors: Klaus WENING, Koln (DE); Lutz BARNSCHEID, Monchengladbach (DE); Anja GEISSLER, Stolberg (DE); Jana DENKER, Bornheim (DE); Dagmar LISCHKE, Inden-Altdorf (DE)

(73) Assignee: GRÜNENTHAL GMBH, Aachen (DE)

(21) Appl. No.: 15/791,508

(22) Filed: Oct. 24, 2017

Related U.S. Application Data
(63) Continuation of application No. PCT/EP2016/025040, filed on Apr. 22, 2016.

(30) Foreign Application Priority Data
Apr. 24, 2015 (EP) 15001225.0

(51) Int. Cl. C07D 489/04

(52) U.S. Cl. CPC C07D 489/04; C07B 2200/13

(57) ABSTRACT

Crystalline salts of 4,5 α-epoxy-3-methoxy-17-methylmorphinan-6-one tartrate (1:1) (hydrocodone bitartrate) are disclosed and three polymorphic forms of these salts are reported. The invention further relates to a pharmaceutical composition comprising such a salt and such a salt as a medicament and for the treatment and/or prevention of pain.

This application is a continuation of International Patent Application No. PCT/EP2016/025040, filed Apr. 22, 2017, which claims foreign priority benefit under 35 U.S.C. §119 of the European Patent Application No. 15001225.0, filed Apr. 24, 2015, the disclosures of which are incorporated herein by reference.

The present invention relates to a crystalline salt of hydrocodone bitartrate, i.e. of 4,5 α-epoxy-3-methoxy-17-methylmorphinan-6-one tartrate (1:1).

The invention further relates to a pharmaceutical composition comprising an effective amount of such a salt and such a salt as a medicament and for the treatment and/or prevention of pain.

Hydrocodone bitartrate is a potent analgesic for the relief of moderate to severe pain. This salt can be administered as an injectable solution, suppository, tablet or extended release tablet.

Document U.S. Pat. No. 4,625,918 B2 describes hydrocodone bitartrate forms which are described to be useful as analgesic agents either in combination with or as replacements for hydrocodone bitartrate.

It would be desirable to have access to further crystalline forms of the hydrocodone bitartrate salt. The present invention has the object of addressing these needs and of providing such additional hydrocodone bitartrate salts.

According to the present invention this object is achieved by a crystalline salt of 4,5 α-epoxy-3-methoxy-17-methylmorphinan-6-one tartrate (1:1), according to formula (I):

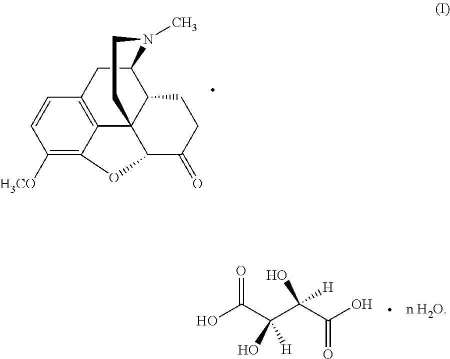

Formula (I) thus describes 4,5 α-epoxy-3-methoxy-17-methylmorphinan-6-one tartrate (1:1), or, in the sense of the present invention, hydrocodone bitartrate.

According to the above formula (I), depending on the respective embodiments, it may be provided that the crystalline salt is present in a hydrate form or in an anhydrous form. Correspondingly, it is provided that 0≦n≦4, preferably 0≦n≦2.5 wherein in the case that n=0, the crystalline form is an anhydrate, whereas in case n>0, the crystalline salt is present in a hydrate form. Regarding the hydrate form, it may be provided that n=2.5, however, it may under circumstances as well be the case that n lies above 0 but below 2.5 or even above 2.5.

The compounds according to the invention are present in a crystalline state which is conveniently defined as the presence of one or preferably more reflexes in an X-ray powder diffractogram (XRPD). In this respect the term “X-ray crystalline” is also used.

It has also been discovered that the crystalline salts of the present invention may be obtained in several polymorphic forms. Polymorphism, the occurrence of different crystal forms, is a property of some molecules and molecular complexes. A single compound may give rise to a variety of polymorphs having distinct crystal structures and physical properties like melting point, x-ray diffraction pattern, infrared absorption fingerprint, and solid state NMR spectrum. One polymorph may give rise to thermal behavior different from that of another polymorph.

The difference in the physical properties of different polymorphs results from the orientation and intermolecular interactions of adjacent molecules or complexes in the bulk solid. Accordingly, polymorphs are distinct solids sharing the same molecular formula yet having distinct advantageous physical properties compared to other polymorphs of the same composition or complex.

The discovery of new polymorphic forms and solvates of a pharmaceutically useful composition provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Therefore, there is a need for additional polymorphs of hydrocodone bitartrate. The crystalline state of an active pharmaceutical substance further has the advantage of an increased stability and processability.

According to the invention, polymorphic forms of hydrocodone bitartrate are provided. Depending on the respective embodiment, the polymorphic forms which are part of the present invention may be provided either in an anhydrous form or as a hydrate. In case the polymorphic forms are provided as a hydrate form, it may be provided that, according to formula (I), n=2.5.

The polymorphic forms according to the present invention may have significant advantages over the prior art and for example over potential amorphous forms of hydrocodone bitartrate.

In fact, the polymorphic forms provided, especially when used as a medicament, may show advantages with regard to efficiency when administered to a patient e.g. due to a good bioavailability as well as with regard to stability when stored. For example, the polymorphic forms of the present invention may show only few or no water absorption. Further, the polymorphic forms provided may have advantages with regard to medicaments showing delayed release.

According to a first embodiment, the X-ray powder diffractogram (XRPD) of the salt comprises the following reflexes (±0.2 in 2θ (2 theta)): 6.9, 11.8, and 12.1. Preferably, the following reflexes (±0.2 in 2θ) are included: 5.5, 6.9, 11.8, 12.1, 13.9 and 14.3. In the context of the present invention this polymorph will be referred to as the “first polymorph”. According to this, the first polymorph preferably is present in a hydrate form, wherein, preferably, with regard to formula (I), n may be 2.5 but may as well be smaller than 2.5.

It is most preferred that its X-ray powder diffractogram comprises one or more of the following reflexes as stated in the following table 1, or it may consist of the following reflexes as stated in the following table 1 (±0.2 in 2θ and recorded using CuK_{α} radiation; the relative intensity I(rel) is 100 at most):

TABLE 1
X-ray powder diffractogram of the first embodiment

| 2θ | I(rel) |
|---|---|
| 5.5 | 8.2 |
| 6.9 | 22.8 |
| 11.8 | 48.1 |
| 12.1 | 35.4 |
| 13.9 | 9.4 |
| 14.3 | 21.3 |

The polymorphic form of hydrocodone bitartrate, i.e. of 4,5 α-epoxy-3-methoxy-17-methylmorphinan-6-one tartrate (1:1) of this first embodiment may be formed as follows:

Firstly, a formulation is provided, which comprises commercially available hydrocodone bitartrate hemi pentahydrate and different further components. The formulation may be formed by weighing and blending the respective compounds. A further step of sieving may further be realized, for example before blending, in order to separate large impurities or agglomerated parts, for example. The formulation may then be extruded by use of an extruder, especially by use of a twin screw extruder. The product being formed in the extruder comprises the first polymorph. Therefore, the first polymorph is present in a formulation.

With regard to the extruder, the latter may comprises different heating zones which are arranged one after the other in a flow direction of the extruded material. For example, a temperature profile may be used which provides a, preferably slight, increase with regard to temperature in the forward direction of the extruded material. The temperature in the extruder may be raised to 100° C. or above, referring to the heating element. Regarding the temperature used, the first heating zone especially at the feeding portion of the extruder may lie in a range of 20° C. to 30° C., wherein the further heating zones may lie in a range of 80° C. to 150° C., wherein, as described above, a temperature increase may be realized.

According to a second embodiment, the X-ray powder diffractogram of the salt comprises one or more of the following reflexes (±0.2 in 2θ): 12.9, 15.0, and 17.1. Preferably, the following reflexes (±0.2 in 2θ) are included: 10.3, 11.3, 12.2, 12.9, 13.8, 15.0, 15.5, 16.2, and 17.1. In the context of the present invention this polymorph according to the second embodiment will be referred to as the “second polymorph”. According to this, the second polymorph preferably is present in a hydrate form, wherein, preferably, with regard to formula (I), n may be 2.5 but may as well be smaller than 2.5.

It is most preferred that its X-ray powder diffractogram comprises one or more of the following reflexes as stated in the following table 2, or it may consist of the following reflexes as stated in the following table 2 (±0.2 in 2θ and recorded using CuK_{α} radiation; the relative intensity I(rel) is 100 at most):

TABLE 2
X-ray powder diffractogram of the second embodiment

| 2θ | I(rel) |
|---|---|
| 10.3 | 7.0 |
| 11.3 | 4.6 |
| 12.2 | 4.3 |
| 12.9 | 19.6 |
| 13.8 | 8.2 |
| 15.0 | 10.6 |
| 15.5 | 5.0 |
| 16.2 | 5.7 |
| 17.1 | 12.9 |

The polymorphic form of hydrocodone bitartrate, i.e. of 4,5 α-epoxy-3-methoxy-17-methylmorphinan-6-one tartrate (1:1) of this second embodiment may be formed as follows:

Firstly, a formulation is provided, which comprises commercially available hydrocodone bitartrate hemi pentahydrate and different further components. The formulation may be formed by weighing and blending the respective compounds. A further step of sieving may further be realized, for example before blending, in order to separate large impurities or agglomerated parts, for example. The formulation may then be extruded by use of an extruder, especially by use of a twin screw extruder. The respective product may then be stored at elevated temperatures, such as in a range of 30° C. to 50° C., for example over a period of one week to three weeks. The product being formed such comprises the second polymorph. Therefore, the second polymorph is present in a formulation.

With regard to the extruder, the latter may comprises different heating zones which are arranged one after the other in a flow direction of the extruded material. For example, a temperature profile may be used which provides a, preferably slight, decrease with regard to temperature in the forward direction of the extruded material. The temperature in the extruder may be raised to 100° C. or above, referring to the heating element of the heating zone with the highest temperature. The first temperature zone may further have a lower temperature then the second temperature zone, which may be heated to 100° C. or above and after which a temperature decrease may follow. Regarding the temperature used, the first heating zone especially at the feeding portion of the extruder may lie in a range of 20° C. to 30° C., wherein the further heating zones, excepting the nozzle, which may have a temperature in the range of 120° C. to 150° C., may lie in a range of 40° C. to 120° C., wherein, as described above, a temperature decrease may be realized.

According to a third embodiment, it may be provided that the X-ray powder diffractogram of the salt comprises one or more of the following reflexes (±0.2 in 2θ): 12.0, 16.1, and 17.6. Preferably, the following reflexes (±0.2 in 2θ) are included: 6.7, 10.5, 11.5, 12.0, 14.1, 15.7, 16.1, 17.6, 19.9. In the context of the present invention this third embodiment of a polymorph will be referred to as the “third polymorph”. According to this, the third polymorph preferably is an anhydrate, i.e. with regard to formula (I), it is provided that n=0.

It is most preferred that its X-ray powder diffractogram comprises one or more of the following reflexes as stated in the following table 3, or it may consist of the following reflexes as stated in the following table 3 (±0.2 in 2θ and recorded using CuK_{α} radiation; the relative intensity I(rel) is 100 at most):

TABLE 3
X-ray powder diffractogram of the third embodiment

| 2θ | I(rel) |
|---|---|
| 6.7 | 3.8 |
| 10.5 | 4.1 |
| 11.5 | 2.2 |
| 12.0 | 14.2 |
| 14.1 | 3.5 |
| 15.7 | 1.8 |
| 16.1 | 13.2 |
| 17.6 | 12.9 |
| 19.9 | 3.0 |

The polymorphic form of hydrocodone bitartrate, i.e. of 4,5 α-epoxy-3-methoxy-17-methylmorphinan-6-one tartrate (1:1) of this third embodiment may be formed as follows:

Firstly, a formulation is provided, which comprises commercially available hydrocodone bitartrate hemi pentahydrate and different further components. The formulation may be formed by weighing and blending the respective compounds. A further step of sieving may further be realized, for example before blending, in order to separate large impurities or agglomerated parts, for example. The formulation may then be extruded by use of an extruder, especially by use of a twin screw extruder. The product being formed in the extruder comprises the third polymorph. Therefore, the third polymorph is present in a formulation.

With regard to the extruder, the latter may comprises different heating zones which are arranged one after the other in a flow direction of the extruded material. For example, a temperature profile may be used which provides a, preferably slight, decrease with regard to temperature in the forward direction of the extruded material. The temperature in the extruder may be raised to 100° C. or above, referring to the heating element of the heating zone with the highest temperature. The first temperature zone may further have a lower temperature then the second temperature zone, which may be heated to 100° C. or above and after which a temperature decrease may follow. Regarding the temperature used, the first heating zone especially at the feeding portion of the extruder may lie in a range of 20° C. to 30° C., wherein the further heating zones, excepting the nozzle, which may have a temperature in the range of 120° C. to 150° C., may lie in a range of 40° C. to 120° C., wherein, as described above, a temperature decrease may be realized.

The salt according to the third polymorph may additionally be prepared in an isolated form. This is called the fourth embodiment. The X-ray powder diffractogram of this fourth embodiment comprises one or more of the following reflexes (±0.2 in 2θ): 12.1, 16.1, and 17.6. Preferably, the following reflexes (±0.2 in 2θ) are included: 6.7, 10.5, 12.1, 12.9, 13.5, 14.2, 16.1, 16.3, 17.1, 17.6, 20.1, 23.2, 24.0, 25.9, 26.8, 30.1. Especially preferred, the following reflexes (±0.2 in 2θ) are included: 6.7, 10.3, 10.5, 12.1, 12.9, 13.5, 13.8, 14.2, 15.0, 15.7, 16.1, 16.3, 17.1, 17.6, 18.6, 19.8, 20.1, 21.3, 22.6, 23.2, 24.0, 24.8, 25.5, 25.9, 26.2, 26.8, 27.1, 27.7, 28.7, 29.0, 29.7, 30.1, 34.7. In particular, the following reflexes (±0,2 in 2θ) are included: 6.7, 7.0, 7.5, 7.9, 10.3, 10.5, 10.7, 11.2, 11.4, 12.1, 12.6, 12.9, 13.5, 13.8, 14.2, 14.5, 15.0, 15.3, 15.7, 16.1, 16.3, 17.1, 17.6, 18.3, 18.6, 19.0, 19.4, 19.8, 20.1, 21.3, 22.6, 23.2, 24.0, 24.8, 25.1, 25.5, 25.9, 26.2, 26.8, 27.1, 27.7, 28.7, 29.0, 29.7, 30.1, 30.9, 31.8, 32.3, 34.7, 35.2, 37.1, 37.7, 38.2. In the context of the present invention this embodiment is again the “third polymorph” According to this, the third polymorph having these peaks is preferably present in an anhydrate form, wherein, preferably, with regard to formula (I), n=0. This third polymorph is present in an isolated form.

With regard to a comparison of the third embodiment, and the fourth embodiment and thus a comparison of the third polymorph in a formulation and in an isolated form, it may be provided that special peaks are present or not and the relative intensities may vary. This may be due to an interaction of components being present in the formulation leading to potential differences of the respective spectra.

It is most preferred that the X-ray powder diffractogram of the fourth embodiment comprises one or more of the following reflexes as stated in the following table 4, or it may consist of the following reflexes as stated in the following table 4 (±0.2 in 2θ and recorded using Cuk_{α} radiation; the relative intensity I(rel) is 100 at most):

TABLE 4
X-ray powder diffractogram of the fourth embodiment

| 2θ | I(rel) |
|---|---|
| 6.7 | 26.7 |
| 7.0 | 5.6 |
| 7.5 | 2.6 |
| 7.9 | 2.3 |
| 10.3 | 11.3 |
| 10.5 | 22.2 |
| 10.7 | 5.9 |
| 11.2 | 4.2 |
| 11.4 | 9.2 |
| 12.1 | 88.4 |
| 12.6 | 3.3 |
| 12.9 | 40.4 |
| 13.5 | 24.7 |
| 13.8 | 11.2 |
| 14.2 | 19.2 |
| 14.5 | 7.8 |
| 15.0 | 14.1 |
| 15.3 | 8.5 |
| 15.7 | 10.9 |
| 16.1 | 100 |
| 16.3 | 19.2 |
| 17.1 | 28.5 |
| 17.6 | 81.8 |
| 18.3 | 7.6 |
| 18.6 | 12.5 |
| 19.0 | 8.4 |
| 19.4 | 5.0 |
| 19.8 | 15.5 |
| 20.1 | 30.3 |
| 21.3 | 11.7 |
| 22.6 | 14.1 |
| 23.2 | 20.8 |
| 24.0 | 30.3 |
| 24.8 | 10.8 |
| 25.1 | 9.2 |
| 25.5 | 13.1 |
| 25.9 | 18.1 |
| 26.2 | 15.6 |
| 26.8 | 18.1 |
| 27.1 | 10.4 |
| 27.7 | 12.5 |
| 28.7 | 11.0 |
| 29.0 | 12.9 |
| 29.7 | 10.4 |
| 30.1 | 20.3 |
| 30.9 | 6.8 |
| 31.8 | 8.0 |
| 32.3 | 6.7 |
| 34.7 | 12.6 |
| 35.2 | 7.4 |
| 37.1 | 6.2 |
| 37.7 | 4.5 |
| 38.2 | 5.3 |

The fourth embodiment of the present invention and thus the third polymorphic form of hydrocodone bitartrate, i.e. of 4,5 α-epoxy-3-methoxy-17-methylmorphinan-6-one tartrate (1:1) in an isolated form may be formed as follows.

Commercially available Hydrocodone bitartrate hemi-pentahydrate was processed by means of VXRPD (Variable temperature Xray Powder Diffraction) as follows. The substance was heated in a diffractometer with the following temperature program. The start temperature was 30° C., the heating rate was set to 10° C./min until an end temperature of 150° C. was reached. When having reached 110° C., the anhydrous form according to the third polymorph and thus the fourth embodiment was formed in an isolated form. The diffractometer used was a STOE & Cie StadiP diffractometer having a HT-unit 0.65LT for the capillaries with image plate detector.

A further aspect of the present invention is a pharmaceutical composition comprising a crystalline salt of 4,5 α-epoxy-3-methoxy-17-methylmorphinan-6-one tartrate (1:1) of formula (I) according to the invention in particular in an effective amount.

The quantity of the crystalline hydrocodone bitartrate amounting to an amount, particularly to an effective amount, of this substance depends substantially on the type of formulation and on the desired dosage during the period of administration. The quantity of the respective compounds to be administered to the patient may vary and is, for example, dependent on the weight or age of the patient and also on the manner of administration, on the indication and on the degree of severity of the illness. The effective amount may under circumstances be comparable to the one being known for the person skilled in the art from generally known hydrocodone bitartrate or may be even less.

Oral formulations may be solid formulations, for example tablets, capsules, pills and pastilles, but oral formulations may also be liquid formulations, for example solutions, suspensions, syrups or elixirs. Liquid and solid formulations also encompass the incorporation of the crystalline, hydrocodone bitartrate into solid or liquid foodstuffs. Furthermore, liquids also encompass solutions for parenteral applications, such as, for example, solutions for infusion or injection.

The crystalline hydrocodone bitartrate can be used directly as powders (micronized particles), granulates, suspensions or solutions, or they may be mixed with other pharmaceutically acceptable ingredients and components and then pulverized, in order then to fill the powders into capsules consisting of hard or soft gelatin, to press tablets, pills or pastilles, or in order to suspend or dissolve the powders in a carrier for the purpose of preparing suspensions, syrups or elixirs. Tablets, pills or pastilles can be provided with a coating after pressing.

Pharmaceutically acceptable ingredients and components for the various types of formulation are known as such. It may, for example, be a question of binding agents such as synthetic or natural polymers, medicinal carriers, lubricating agents, surfactants, sweetening agents and flavoring agents, coating agents, preserving agents, dyestuffs, thickening agents, ancillary agents, antimicrobial agents and carriers for the various types of formulation, as they are generally known in the art.

Examples of suitable binding agents include gum arabic, gum tragacanth, acacia gum, xanthan gum and biodegradable polymers such as homopolyesters or copolyesters of dicarboxylic acids, alkylene diols, polyalkylene glycols and/or aliphatic hydroxycarboxylic acids; homopolyamides or copolyamides of dicarboxylic acids, alkylenediamines and/or aliphatic aminocarboxylic acids; corresponding polyester-polyamide copolymers, polyanhydrides, polyorthoesters, polyphosphazenes and polycarbonates. The biodegradable polymers may be linear, branched or crosslinked. Specific examples are polyglycolic acid, polylactic acid and poly-d,l-lactic/glycolic acid. Other examples of polymers are water-soluble polymers such as, for example, polyoxaalkylenes (polyoxyethylene, polyoxypropylene and mixed polymers thereof), polyacrylamides and hydroxyl-alkylated polyacrylamides, polymaleic acid and esters or amides thereof, polyacrylic acid and esters or amides thereof, polyvinyl alcohol and esters or ethers thereof, polyvinyl imidazole, polyvinyl pyrrolidone and natural polymers, such as chitosan, for example.

Examples of medicinal carriers include phosphates, such as dicalcium phosphate.

Examples of suitable lubricating agents include natural or synthetic oils, fats, waxes or fatty-acid salts such as magnesium stearate.

Surfactants (surface-active agents) may be anionic, cationic, amphoteric or neutral. Examples of useful surfactants include lecithin, phospholipids, octyl sulfate, decyl sulfate, dodecyl sulfate, tetradecyl sulfate, hexadecyl sulfate and octadecyl sulfate, sodium oleate or sodium caprate, 1-acylaminoethane-2-sulfonic acids such as 1-octanoylaminoethane-2-sulfonic acid, 1-decanoylaminoethane-2-sulfonic acid, 1-dodecanoylaminoethane-2-sulfonic acid, 1-tetradecanoylamino-ethane-2-sulfonic acid, 1-hexadecanoylaminoethane-2-sulfonic acid and 1-octadecanoylaminoethane-2-sulfonic acid, bile acids, salts and derivatives thereof, such as, for example, cholic acid, deoxycholic acid, taurocholic acid, taurodeoxycholic acid and sodium glycocholates, sodium caprate, sodium laurate, sodium oleate, sodium lauryl sulfate, sodium cetyl sulfate, sulfated castor oil, sodium dioctyl sulfosuccinate, cocamidopropyl betaine and lauryl betaine, fatty alcohols, cholesterols, glycerin monostearate or distearate, glycerin monooleate or dioleate, glycerin monopalmitate or dipalmitate and polyoxyethylene stearate.

Examples of suitable sweetening agents include sucrose, fructose, lactose and aspartame.

Examples of useful flavoring agents include peppermint, oil of wintergreen or fruit flavor such as cherry or orange flavor.

Examples of suitable coating agents include gelatins, waxes, shellac, sugars and biodegradable polymers.

Examples of preservation agents include methylparaben or propylparaben, sorbic acid, chlorobutanol and phenol.

Examples of ancillary agents include aromatic principles.

Examples of suitable thickening agents include synthetic polymers, fatty acids, fatty-acid salts, fatty-acid esters and fatty alcohols.

Examples of suitable liquid carriers include water, alcohols (ethanol, glycerol, propylene glycol, liquid polyethylene glycols), polytriazines and oils. Examples of solid carriers are talc, aluminas, microcrystalline cellulose, silicon dioxide, aluminium oxide and similar solid substances.

The composition according to the invention may also contain isotonic agents such as, for example, sugars, physiological buffers and sodium chloride.

The composition according to the invention may also be formulated as an effervescent tablet or effervescent powder which decomposes in an aqueous environment, thereby formulating solutions or suspensions for drinking.

The composition according to the invention may also be a formulation with delayed and/or controlled release of the active substance upon contact with body fluids of the gastrointestinal tract, in order to achieve a substantially constant and effective level of the active substance in the blood plasma.

The crystalline hydrocodone bitartrate can also be used together with at least one further pharmaceutical active substance for combination therapies. To this end, at least one further active substance may be additionally dispersed or dissolved in the composition according to the invention.

Yet another aspect of the present invention is a crystalline salt of 4,5 α-epoxy-3-methoxy-17-methylmorphinan-6-one tartrate (1:1) of formula (I), i.e. of hydrocodone bitartrate, according to the invention as a medicament. With further reference to the detailed description above, the medicament to the invention may exist as a liquid, semisolid or solid medicinal form, for example in the form of injection solutions, drops, juices, syrups, sprays, suspensions, tablets, patches, capsules, plasters, suppositories, ointments, creams, lotions, gels, emulsions, aerosols, or in multiparticulate form, for example in the form of pellets or granulates, optionally pressed into tablets, filled in capsules or suspended in a liquid, and may also be administered as such.

In a preferred manner, the medicament according to the invention is suitable for the treatment and/or prevention and/or inhibition of pain, preferentially of acute pain, chronic pain, neuropathic pain or visceral pain.

Finally, the present invention is also directed to a crystalline salt of 4,5 α-epoxy-3-methoxy-17-methylmorphinan-6-one tartrate (1:1) of formula (I) according to the invention for the treatment and/or prevention of pain.

In a particularly preferred manner, the crystalline hydrocodone bitartrate salt according to the invention is suitable for the treatment and/or inhibition of pain, preferentially of acute pain, chronic pain, neuropathic pain or visceral pain; depressions; epilepsy; Parkinson's disease; abuse of alcohol and/or drugs (in particular, nicotine and/or cocaine) and/or medicaments; dependence on alcohol and/or drugs (in particular, nicotine and/or cocaine) and/or medicaments; preferentially for the prophylaxis and/or reduction of withdrawal symptoms in the case of dependence on alcohol and/or drugs (in particular, nicotine and/or cocaine) and/or medicaments; of the development of tolerance phenomena in relation to medicaments, in particular in relation to opioids, or for anxiolysis.

Lastly, the present invention also encompasses a method of treating pain in a subject in need thereof, said method comprising administering to said subject an analgesically effective amount of a crystalline anhydrous oxymorphone hydrochloride according to the invention.

### BRIEF DESCRIPTION OF THE DRAWINGS

The present invention will be further described with reference to the following figures and examples without being limited in any way.

FIG. 1 shows an XRPD diagram of the first polymorph of hydrocodone bitartrate in a formulation;

FIG. 2 shows an XRPD diagram of the second polymorph of hydrocodone bitartrate in a formulation;

FIG. 3 shows an XRPD diagram of the third polymorph of hydrocodone bitartrate anhydrate in a formulation; and

FIG. 4 shows an XRPD diagram of the third polymorph of hydrocodone bitartrate in an isolated form;

FIGS. 1 to 4 show XRPD diagrams of the four embodiments and thus three different polymorphic forms of hydrocodone bitartrate. Regarding the diagrams, they were formed by using copper radiation (1.54060 Copper) and a curved Germanium (111) monochromator.

In detail, the data were generated using a Stoe & Cie X-ray powder diffractometer using CuKα radiation, fitted with a fine focus X-ray tube, set at 40 kV and 40 mA and a Germanium monochromator(111). Scan mode was Debye-Scherrer. Diffracted radiation was detected by a linear PSD in 0.5 steps-30 sec/step and 120 sec/step. Continuous scans were used in the 2θ ranges from 5 to 50.

According to FIG. 1 and thus according to the first polymorph, this form may in an exemplarily and non-limiting manner be generated as follows.

The following formulation was used as defined in table 5:

TABLE 5
Formulation used for forming the first polymorph

| per dose [mg] | Substance | Amount [%] |
|---|---|---|
| 10.00 | Hydrocodone bitartrate | 5.56 |
| 1.44 | Citric acid | 0.80 |
| 25.20 | PEG 6000 | 14.00 |
| 0.36 | Alpha-Tocopherole | 0.20 |
| 143.00 | PEO 7 Mio. | 79.44 |
| 180.00 | | 100.00 |

The formulation according to table 5 thus comprises the following ingredients: hydrocodone bitartrate hemi pentahydrate, or, in other words, hydrocodone hydrogentartrate pentahemihydrate commercially available from the company Noramco, citric acid, polyethyleneglycole with an average molecular weight Mn of 6000 (PEG 6000), alpha tocopherole and polyethyleneoxide with an average molecular weight Mn of 7 million (PEO 7 Mio.)

The formulation according to table 5 was processed as follows.

The extrudates were manufactured in a batch size of about 500 g using the process steps weighing, sieving, blending, extrusion. The weighing was performed on a 1 kg balance. Subsequently the powder was sieved using a sieve with a mesh size of 1.0 mm and blended in a Bohle LM40 MC1 blender (12 rpm, 10 min). The final powder blend was then extruded using a Leistritz ZSE 18 (Twin Screw Extruder).

The extruder comprises an extrusion die (nozzle) diameter of 0.8 mm and comprises nine different heating zones and a die which could be separately heated. The speed of the screw or of the two screws may be adjusted to 80-120 rpm, for example to 100 rpm. The feed rate used may be in the range of 10-20 g/min, for example at 16.66 g/min. The formulation was extruded to result in pellets having a diameter in a range of 0.8-1.2 mm, particularly of 0.8 mm.

The temperature in the extruder was steadily increased from room temperature at the entry to 136° C. at the exit, whereas the first heating zone was adjusted to a temperature within the range of from 20° C. to 30° C. and the following heating zones were each adjusted to temperatures within the range of from 80° C. to 136° C. in a manner such that a steady temperature increase was realized.

This polymorphic form, or its X-ray powder diffractogram, respectively, according to FIG. 1, comprises one or more of the following reflexes as stated in the following table 6, or it may consist of the following reflexes as stated in the following table 6 (±0.2 in 2θ and recorded using CuK_{α} radiation; the relative intensity I(rel) is 100 at most):

TABLE 6
X-ray powder diffractogram of the first embodiment

| 2θ | I(rel) |
|---|---|
| 5.5 | 8.2 |
| 6.9 | 22.8 |
| 11.8 | 48.1 |
| 12.1 | 35.4 |
| 13.9 | 9.4 |
| 14.3 | 21.3 |

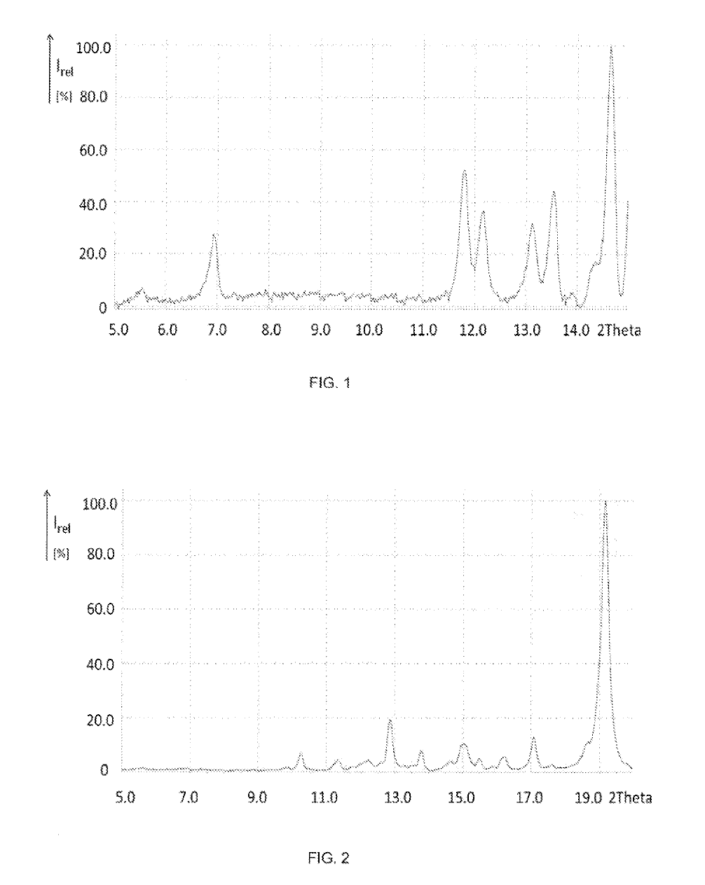

According to FIG. 2 and thus according to the second polymorph, this form may in an exemplarily and non-limiting manner be generated as follows.

The following formulation was used as defined in table 7:

TABLE 7
Formulation used for forming the second polymorph

| per dose [mg] | Substance | Amount [%] |
|---|---|---|
| 50.0 | Hydrocodone bitartrate | 18.6 |
| 2.3 | Citric acid | 0.8 |
| 26.9 | Hypromellose 100000 mPa*s | 10.0 |
| 36.4 | PEG 6000 | 13.6 |
| 0.5 | Alpha-Tocopherole | 0.2 |
| 152.7 | PEO 7 Mio. Dow | 56.8 |
| 268.8 | | 100.00 |

The formulation according to table 7 thus comprises the following ingredients: hydrocodone bitartrate hemi pentahydrate, or, in other words, hydrocodone hydrogentartrate pentahemihydrate commercially available from the company Noramco, citric acid, hydroxypropyl methycellulose with a viscosity of 100000 mPa*s (Hypromellose 100000 mPa*s), polyethyleneglycole with an average molecular weight Mn of 6000 (PEG 6000), alpha tocopherole and polyethyleneoxide with an average molecular weight Mn of 7 million (PEO 7 Mio.), available by the company Dow chemicals.

The formulation according to table 7 was processed as follows.

The extrudates were manufactured in a batch size of about 500 g using the process steps weighing, sieving, blending, extrusion. The weighing was performed on a 1 kg balance. Subsequently the powder was sieved using a sieve with a mesh size of 1.0 mm and blended in a Bohle LM40 MC1 blender (12 rpm, 10 min). The final powder blend was then extruded using a Leistritz ZSE 18 (Twin Screw Extruder).

The extruder comprises an extrusion die (nozzle) diameter of 5 mm and comprises nine different heating zones and a die which could be separately heated. The speed screw may be adjusted in 80-12 rpm, for example to 100 rpm. The feed rate used may be in the range of 10-20 g/min, for example at 16.66 g/min. The formulation was extruded to result in a strand having a diameter in a range of 5 mm and having a length of 9.3 mm.

The temperature in the extruder was varied from room temperature at the entry to 135° C. at the exit. The first heating zone was adjusted to a temperature within the range of from 20° C. to 30° C., whereas the further heating zones, except the nozzle, which had a temperature of 135° C., were each adjusted to temperatures within the range of from 40° C. to 120° C. in such a manner that an initial temperature increase and a subsequent intermediate temperature decrease was realized.

Subsequently, the product was stored at 40° C. for two weeks at 75% relative humidty (% rH).

This polymorphic form or its X-ray powder diffractogram according to FIG. 2, comprises one or more of the following reflexes as stated in the following table 8, or it may consist of the following reflexes as stated in the following table 8 (±0.2 in 2θ and recorded using CuK_{α} radiation; the relative intensity I(rel) is 100 at most):

TABLE 8
X-ray powder diffractogram of the second polymorph

| 2θ | I(rel) |
|---|---|
| 10.3 | 7.0 |
| 11.3 | 4.6 |
| 12.2 | 4.3 |
| 12.9 | 19.6 |
| 13.8 | 8.2 |
| 15.0 | 10.6 |
| 15.5 | 5.0 |
| 16.2 | 5.7 |
| 17.1 | 12.9 |

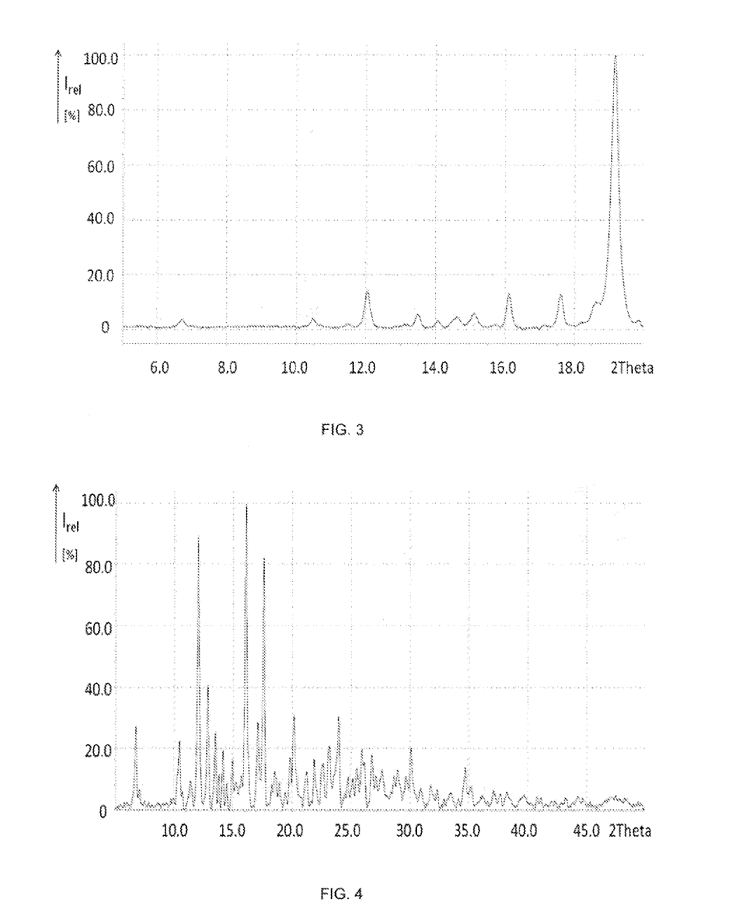

According to FIG. 3 and thus according to the third polymorph, this form may in an exemplarily and non-limiting manner be generated as follows.

The following formulation was used as defined in table 9:

TABLE 9
Formulation used for forming the third polymorph

| per dose [mg] | Substance | Amount [%] |
|---|---|---|
| 46.00 | Hydrocodone bitartrate | 17.11 |
| 2.30 | Citric acid | 0.86 |
| 27.60 | Hypromellose 100000 mPa*s | 10.27 |
| 37.30 | PEG 6000 | 13.88 |
| 0.55 | Alpha-Tocopherole | 0.20 |
| 155.05 | PEO 7 Mio. Sumitomo | 57.68 |
| 268.80 | | 100.00 |

The formulation according to table 9 thus comprises the following ingredients: hydrocodone bitartrate hemi pentahydrate, or, in other words, hydrocodone hydrogentartrate pentahemihydrate commercially available from the company Noramco, citric acid, hydroxypropyl methycellulose with a viscosity of 100000 mPa*s (Hypromellose 100000 mPa*s), polyethyleneglycole with an average molecular weight Mn of 6000 (PEG 6000), alpha tocopherole and polyethyleneoxide with an average molecular weight Mn of 7 million (PEO 7 Mio.), available by the company Sumitomo.

The formulation according to table 9 was processed as follows.

The extrudates were manufactured in a batch size of about 500 g using the process steps weighing, sieving, blending, extrusion. The weighing was performed on a 1 kg balance. Subsequently the powder was sieved using a sieve with a mesh size of 1.0 mm and blended in a Bohle LM40 MC1 blender (12 rpm, 10 min). The final powder blend was then extruded using a Leistritz ZSE 18 (Twin Screw Extruder).

The extruder comprises an extrusion die (nozzle) diameter of 5 mm and comprises nine different heating zones and a die which could be separately heated. The speed screw may be adjusted in 80-12 rpm, for example to 100 rpm. The feed rate used may be in the range of 10-20 g/min, for example at 16.66 g/min. The formulation was extruded to result in a strand having a diameter in a range of 5 mm and having a length of 8.5 mm.

The temperature in the extruder was varied from room temperature at the entry to 135° C. at the exit. The first heating zone was adjusted to a temperature within the range of from 20° C. to 30° C., whereas the further heating zones, except the nozzle, which had a temperature of 135° C., were each adjusted to temperatures within the range of from 40° C. to 120° C. in such a manner that an initial temperature increase and a subsequent intermediate temperature decrease was realized.

It is most preferred that its X-ray powder diffractogram comprises one or more of the following reflexes as stated in the following table 10, or it may consist of the following reflexes as stated in the following table 10 (±0.2 in 2θ and recorded using CuK_{α} radiation; the relative intensity I(rel) is 100 at most):

TABLE 10
X-ray powder diffractogram of the third embodiment

| 2θ | I(rel) |
|---|---|
| 6.7 | 3.8 |
| 10.5 | 4.1 |
| 11.5 | 2.2 |
| 12.0 | 14.2 |
| 14.1 | 3.5 |
| 15.7 | 1.8 |
| 16.1 | 13.2 |
| 17.6 | 12.9 |
| 19.9 | 3.0 |

According to FIG. 4 and thus according to the fourth embodiment referring to an isolated form of the third polymorphic form of hydrocodone bitartrate, i.e. of 4,5 α-epoxy-3-methoxy-17-methylmorphinan-6-one tartrate (1:1), this form may in an exemplarily and non-limiting manner be generated as follows. hydrocodone bitartrate hemi pentahydrate, or, in other words, hydrocodone hydrogentartrate pentahemihydrate commercially available from the company Noramco, was processed by means of VXRPD (Variable temperature Xray Powder Diffraction) as follows. The substance was heated in a diffractometer with the following temperature program. The start temperature was 30° C., the heating rate was set to 10° C./min until an end temperature of 150° C. was reached. When having reached 110° C., the anhydrous form according to the fourth embodiment was formed. The diffractometer used was a STOE & Cie StadiP diffractometer having a HT-unit 0.65LT for the capillaries.

It is most preferred that its X-ray powder diffractogram comprises one or more of the following reflexes as stated in the following table 11, or it may consist of the following reflexes as stated in the following table 11 (±0.2 in 2θ and recorded using CuK_{α} radiation; the relative intensity I(rel) is 100 at most).

TABLE 11
X-ray powder diffractogram of the fourth embodiment

| 2θ | I(rel) |
|---|---|
| 6.7 | 26.7 |
| 7.0 | 5.6 |
| 7.5 | 2.6 |
| 7.9 | 2.3 |
| 10.3 | 11.3 |
| 10.5 | 22.2 |
| 10.7 | 5.9 |
| 11.2 | 4.2 |
| 11.4 | 9.2 |
| 12.1 | 88.4 |
| 12.6 | 3.3 |
| 12.9 | 40.4 |
| 13.5 | 24.7 |
| 13.8 | 11.2 |
| 14.2 | 19.2 |
| 14.5 | 7.8 |
| 15.0 | 14.1 |
| 15.3 | 8.5 |
| 15.7 | 10.9 |
| 16.1 | 100 |
| 16.3 | 19.2 |
| 17.1 | 28.5 |
| 17.6 | 81.8 |
| 18.3 | 7.6 |
| 18.6 | 12.5 |
| 19.0 | 8.4 |
| 19.4 | 5.0 |
| 19.8 | 15.5 |
| 20.1 | 30.3 |
| 21.3 | 11.7 |
| 22.6 | 14.1 |
| 23.2 | 20.8 |
| 24.0 | 30.3 |
| 24.8 | 10.8 |
| 25.1 | 9.2 |
| 25.5 | 13.1 |
| 25.9 | 18.1 |
| 26.2 | 15.6 |
| 26.8 | 18.1 |
| 27.1 | 10.4 |
| 27.7 | 12.5 |
| 28.7 | 11.0 |
| 29.0 | 12.9 |
| 29.7 | 10.4 |
| 30.1 | 20.3 |
| 30.9 | 6.8 |
| 31.8 | 8.0 |
| 32.3 | 6.7 |
| 34.7 | 12.6 |
| 35.2 | 7.4 |
| 37.1 | 6.2 |
| 37.7 | 4.5 |
| 38.2 | 5.3 |

1. A crystalline, salt of 4,5 α-epoxy-3-methoxy-17-methylmorphinan-6-one tartrate (1:1), according to formula (I): 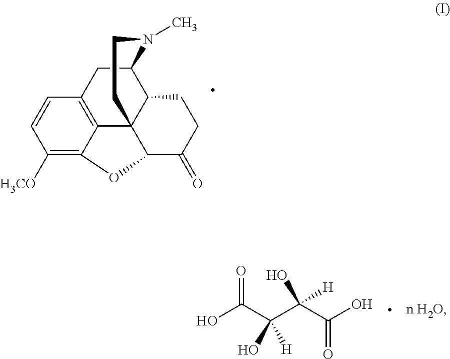
wherein 0≧n≧4, and wherein the X-ray powder diffractogram of the salt comprises one or more of the following reflexes (±0.2 in 2θ): 6.9, 11.8, and 12.1, or wherein the X-ray powder diffractogram of the salt comprises one or more of the following reflexes (±0.2 in 2θ): 12.9, 15.0, and 17.1, or wherein the X-ray powder diffractogram of the salt comprises one or more of the following reflexes (±0.2 in 2θ): 12.0, 16.1, and 17.6.

2. The salt according to claim 1, wherein the X-ray powder diffractogram of the salt comprises one or more of the following reflexes (±0.2 in 2θ): 5.5, 6.9, 11.8, 12.1, 13.9 and 14.3.

3. The salt according to claim 1, wherein the X-ray powder diffractogram of the salt comprises one or more of the following reflexes (±0.2 in 2θ): 10.3, 11.3, 12.2, 12.9, 13.8, 15.0, 15.5, 16.2, 17.1.

4. The salt according to claim 1, wherein the X-ray powder diffractogram of the salt comprises one or more of the following reflexes (±0.2 in 2θ): 6.7, 10.5, 11.5, 12.0, 14.1, 15.7, 16.1, 17.6, 19.9.

5. The salt according to claim 1, wherein the X-ray powder diffractogram of the salt comprises one or more of the following reflexes (±0.2 in 2θ): 6.7, 10.5, 12.1, 12.9, 13.5, 14.2, 16.1, 16.3, 17.1, 17.6, 20.1, 23.2, 24.0, 25.9, 26.8, 30.1.

6. The salt according to claim 5, wherein the X-ray powder diffractogram of the salt comprises one or more of the following reflexes (±0.2 in 2θ): 6.7, 10.3, 10.5, 12.1, 12.9, 13.5, 13.8, 14.2, 15.0, 15.7, 16.1, 16.3, 17.1, 17.6, 18.6, 19.8, 20.1, 21.3, 22.6, 23.2, 24.0, 24.8, 25.5, 25.9, 26.2, 26.8, 27.1, 27.7, 28.7, 29.0, 29.7, 30.1, 34.7.

7. The salt according to claim 6, wherein the X-ray powder diffractogram of the salt comprises one or more of the following reflexes (±0.2 in 2θ): 6.7, 7.0, 7.5, 7.9, 10.3, 10.5, 10.7, 11.2, 11.4, 12.1, 12.6, 12.9, 13.5, 13.8, 14.2, 14.5, 15.0, 15.3, 15.7, 16.1, 16.3, 17.1, 17.6, 18.3, 18.6, 19.0, 19.4, 19.8, 20.1, 21.3, 22.6, 23.2, 24.0, 24.8, 25.1, 25.5, 25.9, 26.2, 26.8, 27.1, 27.7, 28.7, 29.0, 29.7, 30.1, 30.9, 31.8, 32.3, 34.7, 35.2, 37.1, 37.7, 38.2.

8. A pharmaceutical composition comprising a crystalline salt of 4,5 α-epoxy-3-methoxy-17-methylmorphinan-6-one tartrate (1:1) of formula (I) according to claim 1.

9. A method of treating and/or preventing pain in a patient in need thereof, said method comprising administering to said patient an effective amount therefor of a crystalline salt of 4,5 α-epoxy-3-methoxy-17-methylmorphinan-6-one tartrate (1:1) of formula (I) according to claim 1.
